(19) United States
(12) Patent Application Publication
(10) Pub. No.: US 2013/0302428 A1
(43) Pub. Date: Nov. 14, 2013

(54) PROCESS FOR PREPARING CARBON PROTECTED SUPERPARAMAGNETIC OR MAGNETIC NANOSPHERES

(71) Applicants: Ferdi Schueth, Mulheim (DE); Mathias Feyen, Weinheim (DE); An-Hui Lu, Dalian City (CN); Wen-Cui Li, Dalian City (CN); Guang-Hui Wang, Mulheim an der Ruhr (DE); Tao Sun, Dalian City (CN)

(72) Inventors: Ferdi Schueth, Mulheim (DE); Mathias Feyen, Weinheim (DE); An-Hui Lu, Dalian City (CN); Wen-Cui Li, Dalian City (CN); Guang-Hui Wang, Mulheim an der Ruhr (DE); Tao Sun, Dalian City (CN)

(73) Assignees: DALIAN UNIVERSITY OF TECHNOLOGY, Liaoning (CN); STUDIENGESELLSCHAFT KOHLE MBH, Mülheim an der Ruhr (DE)

(21) Appl. No.: 13/884,352

(22) PCT Filed: Nov. 9, 2011
(86) PCT No.: PCT/EP2011/069719

§ 371 Date: Jul. 22, 2013

(30) Foreign Application Priority Data
Sep. 11, 2010 (DE) 10 2010 050 644.3

(51) Int. Cl. H01F 1/01; H01F 41/00; A61K 9/14

(52) U.S. Cl. CPC H01F 1/01; A61K 9/14; H01F 41/00
USPC 424/490; 423/274; 252/62.56; 502/439; 427/127

(57) ABSTRACT

A process for the preparation of carbon protected superparamagnetic or magnetic nanospheres is claimed which comprises the steps:

(A) coating FeₓO_{y} particles with an organic polymer,
(B) coating the product obtained in step (A) with silica,
(C) subjecting the product of step (B) to pyrolysis conditions, and
(D) removing silica.

According to present process, structurally stable carbon protected magnetic nanospheres were obtained, which can be dispersed in various solvents like water, EtOH, toluene, etc.

This application is a 371 of International Patent Application No. PCT/EP2011/069719, filed Nov. 9, 2011, which claims foreign priority benefit under 35 U.S.C. §119 of the German Patent Application No. 10 2010 050 644.3 filed Nov. 9, 2010, the disclosures of which are incorporated herein by reference.

The present invention relates to a process for preparing carbon protected superparamagnetic or magnetic nanospheres, carbon protected superparamagnetic or magnetic nanospheres obtainable by such process and the use of the nanospheres in the catalysis, as magnetic fluids and as transport media in drug targeting and contrast agents in imaging methods.

Magnetic nanoparticles are of great interest for catalysis, magnetic fluid, biotechnology/biomedicine and so on. One big encountered problem is that magnetic nanoparticles have the tendency to cluster and precipitate, which dramatically reduces their efficiency. Therefore, the surfaces of these magnetic nanoparticles need to be passivated by organic or inorganic coatings, to minimize the agglomeration and oxidation, thus making the nanoparticles dispersible and stable in a variety of media.

Up to date, magnetic nanoparticles can be coated with surfactant, polymer, or silica, to maintain their dispersibility. However, surfactant and polymer coated magnetic nanoparticles cannot survive at temperatures exceeding 150° C., because the metallic nanoparticles can catalyze the decomposition of the attached polymer to form other species, which results in destruction of the protection shell, and corresponding loss of the magnetization of the nanoparticles. For silica coated magnetic nanoparticles, it is difficult to achieve a really dense and non-porous silica coating layer, it is thus difficult to maintain their stability under harsh conditions, such as strong acid and base conditions. Therefore, it is scientifically and technologically important to explore synthetic method for the preparation of highly stable magnetic nanoparticles which are stable at high temperature, strong acid and base conditions. This guarantees the long-life and safe utilization of magnetic nanoparticles in catalysis and biomedicine.

To protect magnetic nanoparticles from oxidation or erosion, carbon materials are among the most suitable candidate as the protective shell because of their thermal stability, chemical resistance and biocompatibility. Hence, the main goal of this invention is to synthesize isolated, highly stable, and carbon protected magnetic nanoparticles.

From the state of art nanoparticles having a core-shell structure are known.

Sun et al. disclose in Chem. Mater. 2006, 18, 3486-3944 a method for the preparation of oxide core-shell nanostructures with carbonaceous polysaccharide shells and oxide (including hydroxides or complex oxides) cores. The oxides are dispersed in an aqueous glucose solution, the suspension is transferred into autoclaves and kept at 180 degrees. From this process nanoparticles having different structures, like rods and plates, can be encapsulated in amorphous carbonaceous shells.

Seo et al. describe in Nature Materials, Vol. 5, December 2006, 971-976 the preparation of FeCo/graphitic-shell nanocrystals as magnetic-resonance-imaging and near-infrared agents. For the preparation of FeCo/graphitic carbon nanocrystals fumed silica is impregnated with methanolic solutions of Fe and Co salts, the dried impregnated silica is then subjected to methane chemical CVD. The obtained product is treated with HF in order to remove the silica.

In US 2009/0047220 a contrast medium for administration to a patient for magnetic resonance imaging is disclosed, the contrast medium comprises a plurality of carbon nanospheres and iron containing nanoparticle embedded in each of the nanospheres.

The core-shell particles obtained according to the state of art are structures of different nature, such as plates etc., spherical structures are difficult to obtain. The literature further shows that carbonaceous nanospheres containing a core of a magnetic oxide are only obtainable by using toxic substances like HF. There is a permanent requirement for improved processes for the preparation of carbon protected oxide nanospheres.

The subject matter of the present invention is therefore a process for the preparation of carbon protected superparamagnetic or magnetic nanospheres comprising the steps:

(A) coating magnetic and/or superparamagnetic particles with an organic polymer,
(B) coating the product obtained in step (A) with silica,
(C) subjecting the product of step (B) to pyrolysis conditions, and
(D) removing silica.

A schematic illustration of the synthetic concept for the synthesis of the carbon protected nanospheres according to the present invention is shown in FIG. 1. According to the process of the present invention, structurally stable carbon protected magnetic nanospheres were obtained, which can be dispersed in various solvents like water, EtOH, toluene, etc. The carbon coating components can be further modified with, for instance carboxyl groups, —NH₂ groups, or others, providing the possibility to covalently binding organic entities, or adsorbing such or other entities by electrostatic interactions. This kind of magnetic nanoparticles is promising for applications in catalysis, biotechnology/biomedicine, etc.

The nanospheres obtained according to the process of the present invention are discrete, structurally stable, carbon protected magnetic nanospheres having permanent magnetic or superparamagnetic properties. The nanospheres show long term stability in acidic and base solutions. The carbon shell can be amorphous and/or graphitic and has a high surface area between 100 and 1.000 m²/g. The sphere sizes may vary from 60 nm to 1 μm. The particles form stable suspensions in water, ethanol, toluene and other organic solvents. They are magnetically separable and tunable in magnetic core and magnetization. As compared to silica coated or polymer coated materials, the carbon protected materials are much more stable, dispersible in many media. Furthermore, easy size control and magnetic core control (and thus the magnetization) is possible.

In step (A) of the process of the present invention, magnetic and/or superparamagnetic nanoparticles are coated with an organic polymer. The nanoparticles may be obtained according to synthesis procedures known from the state of the art. In case, in step A Fe oxides are used as nanoparticles, these oxides may be prepared by a precipitation procedure wherein salts of Fe (II) and/or of Fe (III) are dissolved in an aqueous solution and reacted with a base, for example ammonium hydroxide or an alkali hydroxide. After the precipitation reaction the obtained oxides may be stabilized by adding a surfactant, a fatty acid, or other stabilizing agents. Examples for suitable fatty acids are carbonic acids having preferably 8 to 22 carbon atoms for example oleic acid, stearic, lauric, linoleic, linolenic, arachidonic, etc. and any mixtures thereof.

The nanoparticles used in step (A) may be selected from any magnetic or superparamagnetic materials. Preferably they are selected from magnetic or superparamagnetic metals and/or metal compounds such as Fe, Co, Ni, Mn, Pd, Cr, and any compounds and mixtures thereof. Preferably, Fe and FeₓO_{y} are used as magnetic and/or superparamagnetic nanoparticles.

The nanoparticles used in step (A) with an average particle size from 1 to 300, more preferably from 5 to 250 nm. For example, α- and γ-Fe₂O₃ nanoparticles with particle sizes ranging from 20-200 nm are also suitable as the magnetic cores for further polymer coating. The stabilization of the nanoparticles has the advantage of preventing the nanoparticles from aggregation.

Coating of the nanoparticles with the organic polymer can be affected by any method known to men skilled in the art. Preferably the polymer is deposited on the surface of the nanoparticles by reacting one or more precursor components of the polymer in the presence of the nanoparticles. The polymerisation reaction may be preferably a polycondensation or radical initiated polymerization such as a polyaddition of the precursor component(s).

The precursor of the polymer may be preferably selected from the group consisting of aromatic compounds which can polymerized with aldehydes. Other polymer precursors which are suitable for coating surfaces, such as hexamethylene tetramine, styrene, divinylbenzene(meth)acrylates, glycidyl(meth)acrylate(s), a mixture of styrene, divinylbenzene, (meth)acrylate and glycidyl(meth)acrylate are also applicable. The aromatic compounds such as phenol, resorcinol, phlorogrucinol, dihydroxybenzoic acid, and aldehydes such as formaldehyde, acetaldehyde, propaldehyde, glutaraldehyde are especially preferred. The coating step (A) is preferably carried out in the presence of a solvent or solvent mixture, i.e. the reaction mixture of step (A) is present as a suspension or dispersion. The presence of the nanoparticles to be coated in the form of a suspension or dispersion has the advantage, that the particles may be prevented from aggregation, and in the end product the cores, consisting of magnetic or superparamagnetic particles, are nanosized. Any solvent which does not adversely affect the process may be used, such as water and organic solvents or mixtures of water and solvents that are miscible with water, such as alcohols.

The particles obtained from step (A) are spherical and have a core of magnetic or superparamagnetic nanoparticles and a polymer shell. The sizes of these particles are approximately 20 nm to 1000 nm and preferably the particle sizes are from 50 nm to 500 nm. Most preferably, the particle sizes are from 80 nm to 300 nm.

The polymer coated particles obtained in (A) are coated in step (B) with silica. This coating step may be carried out by any process known by men skilled in the art. In the preferred process one or more precursor(s) of silica are subjected to hydrolysis conditions in the presence of the polymer coated particles obtained in step (A). The precursors of silica which form silica under hydrolysis conditions are known in the art. Preferred examples are silanes of the general formula (R¹O)₄—Si, wherein R¹ is selected from an alkyl group having 1 to 6 carbon atoms. Among the siliane compounds, TMOS and TEOS are most preferred.

The hydrolysis can be accelerated by carrying out the hydrolysis under basic conditions, preferably at a pH of 8 or higher. As a base ammonia solution or an aqueous solution of alkali hydroxide may be used for adjusting the pH value. The obtained nanospheres having a SiO₂ coating as the outer shell may be separated from the solution by any manner known for this, for example by filtration or centrifugation. The product of step (B) may be washed and/or dried before it is further processed, or it can be used as it is for the next step.

In process step (C) the polymer shell is converted into carbon. For carrying out step (C) the product obtained in step (B) is subjected to pyrolysis conditions. The pyrolysis is carried out at a temperature which is high enough between 200° C. and 1100° C. in order to convert the polymer shell into a carbon shell, and preferably pyrolysis is performed at a temperature of between 400° C. and 850° C. Most preferably, the pyrolysis is performed at a temperature between 500° C. and 700° C. The pyrolysis may be carried out by any method known in the state of art. In order to prevent shrinkage or any other reaction of the carbon shell which would occur by the reaction of the carbon with O₂ in the surrounding, the pyrolysis is preferably carried out under inert gas atmosphere. In step (C) nanospheres are obtained having one or more cores of magnetic or superparamagnetic particles, an inner shell of carbon and an outer shell of silica.

The removal of the silica may be effected by dissolving silica, for example by dissolving silica in a basic solution having a pH between 10 and 14, or more basic solution.

The particles obtained in the process of present invention according to steps (A) to (D) are carbon-protected monodisperse nanospheres showing superparamagnetic or magnetic properties. The magnetic properties and the structurally properties are shown in the examples enclosed herewith.

The carbon protected superparamagnetic or magnetic nanospheres obtained according to the process of present invention are useful as catalytic particles, in magnetic fluids, and in biotechnology/biomedicine, such as contrast agents in imaging methods or for drug targeting. The particles are especially useful in biotechnology/biomedicine processes such as hyperthermia, separation of biomolecules and enrichment of biomolecules.

### BRIEF DESCRIPTION OF THE DRAWINGS

The invention is further illustrated in FIGS. 1 to 13, wherein:

FIG. 1 represents a schematic illustration of the synthetic principle of discrete carbon protected nanospheres comprising the steps of:

(1) Providing magnetic nanoparticles, (2) polymer encapsulation, (3) silica coating, (4) pyrolysis, (5) removal of silica;

FIG. 2a-2d show TEM images of PFM-2, PFM-3, PFM-4 and PFM-7;

FIG. 3 shows the effect of the amount of Fe₃O₄ nanoparticles on the size of Fe₃O₄@PF, wherein:

—— The size of Fe₃O₄/PF nanospheres
—▪— The size of multi-core spheres;

FIGS. 4a-4f show TEM images of PFM-1@SiO₂ (a), PFM-4@SiO₂ (b), and PFM-7@SiO₂ (c); the corresponding TEM images at high magnification (d, e, f);

FIGS. 5a-5f show TEM image (a), SEM image (b), and STEM image (c) of PFM-1-600; TEM image (d), SEM image (e), and STEM image (f) of PFM-1-800;

FIGS. 6a-6d show TEM images of PFM-1-500 (a), PFM-1-600 (b), PFM-1-700 (c) and PFM-1-800 (d) after concentrated hydrochloric acid treatment for 7 days at room temperature;

FIG. 7 shows magnetization curves for the FeₓO_{y}@C obtained from different pyrolyzed temperature and after concentrated HCl washing;

FIGS. 8a-8d show TEM images of Fe₂O₃@PF (a), Fe₂O₃@PF@MSiO₂ (b), Fe₂O₃@PF@MSiO₂ (c) (600° C. carbonization), and FeₓO_{y}@C (d);

FIGS. 9a-9d show TEM image of FeₓO_{y}@C obtained from different pyrolyzed temperature: 500° C. (a), 600° C. (b), 700° C. (c), and 800° C. (d);

FIG. 10 shows magnetization curves for the FeₓO_{y}@C obtained from different pyrolyzed temperature and after concentrated HCl washing;

FIG. 11 shows a XRD pattern of FeₓO_{y}@C pyrolyzed at 600° C. and after concentrated HCl washing;

FIG. 12 shows a XRD pattern of FeₓO_{y}@C pyrolyzed at 800° C. and after concentrated HCl washing; and

FIGS. 13.1-13.4 show TEM images of Fe₃O₄@HDA 1 (a), Fe₂O₃@HDA 1(b), Fe₃O₄@PSty 2(a), Fe₂O₃@PSty 2(b), Fe₃O₄@PSty@SiO₂ 3(a), Fe₂O₃@PSty@SiO₂ 3(b), Fe@C (100 nm) 4(a), Fe@C (200 nm) 4(b), Fe@C (20 nm) 4(c), and Fe@C (50 nm) 4(d).

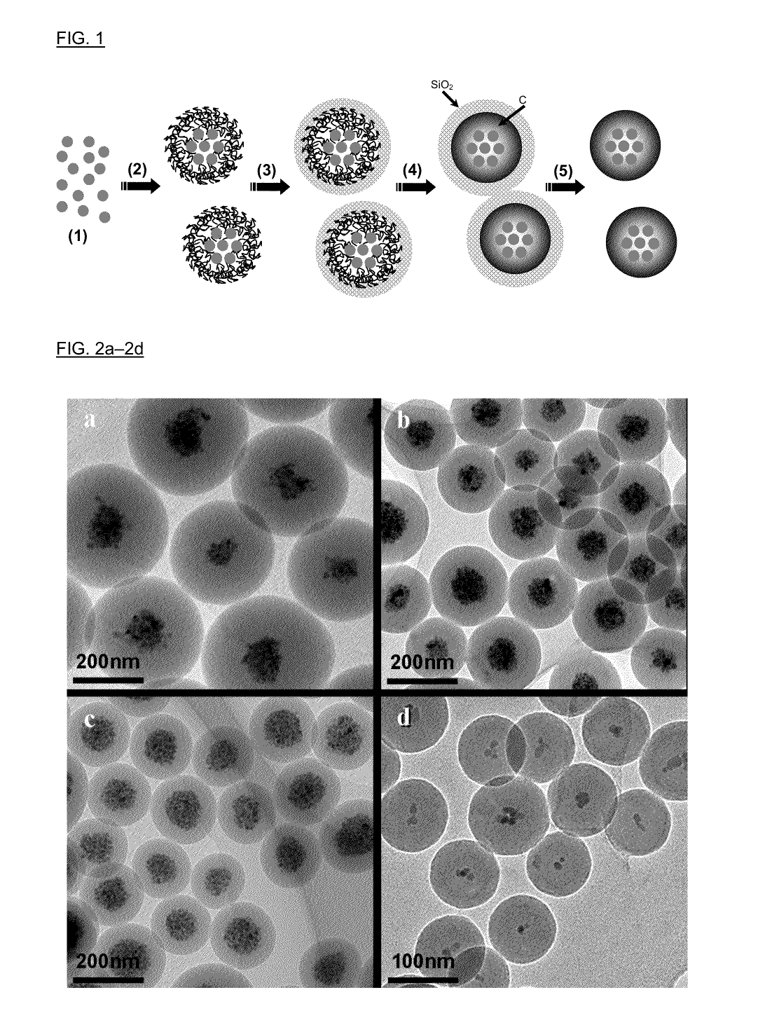

The invention is further explained in the following examples.

### 1. Examples for Fe₃O₄ Based Nanoparticles

### 1.1 Synthesis of Fe₃O₄ Nanoparticles

Fe₃O₄ nanoparticles stabilized by oleic acid with an average particle size ˜10 nm were synthesized by a modified chemical coprecipitation method. Typically, 1 g FeCl₃.6H₂O, 0.409 g FeCl₂.4H₂O and 0.052 g F127 were dissolved in 50 ml deionized water under nitrogen gas with vigorous stirring at 80° C. Then 1.8 ml of ammonium hydroxide was added rapidly into the solution. The colour of solution turned to black immediately. After reaction for 30 minutes, 0.35 ml of oleic acid was added into the solution and kept reacting at 80° C. for 1 hour. Finally, the stable colloid solution containing magnetite nanoparticles stabilized by oleic acid was obtained.

### 1.2 Synthesis of Fe₃O₄@PF Nanospheres

In a typical procedure, 1 ml of the solution of Fe₃O₄@oleic acid obtained in step 1.1 (≈0.04 mmol Fe₃O₄) was ultrasonicated for 10 min in 25 ml of 1 M HCl solution. Then, Fe₃O₄ magnetic nanoparticles were collected with the help of a magnet and washed two times with deionized water. The collected Fe₃O₄ magnetic nanoparticles were redispersed in 20 ml of deionized water and maintained at 80° C. for 30 min. 80 ml of aqueous solution containing 1.25 mmol phenol and 0.625 mmol HMT was added into the above solution followed by ultrasonication for 30 min at 50° C. The mixed solution was transferred to a Teflon-lined stainless steel autoclave of 150 ml capacity, sealed, and maintained at 160° C. for 4 hours. Afterwards, the autoclave was allowed to cool down to room temperature. The products were collected by centrifugation at 8000 rpm for 10 min, washed three times with deionized water and once with absolute ethanol, and finally dried in an oven at 50° C. for 8 h. The shell thickness of the Fe₃O₄@PF can be controlled by the amount of Fe₃O₄ nanoparticles. Increasing the amount of Fe₃O₄ nanoparticles and maintaining the other reaction conditions constant, the shell thickness decreased. The final products were denoted as PFM-x, where x indicates the sample number of the polymer composites (See Table 1).

TABLE 1
Examples: experimental conditions for the preparation
of Fe₃O₄@PF nanospheres.

| | | | | Sodium |
|---|---|---|---|---|
| | Fe₃O₄ | Phenol/HMT | | oleate |
| Sample | [mmol] | [mmol] | Temp(° C.)/Time(h) | [mg] |
| PFM-1 | 0.04 | 1.25/0.625 | 160/4 | — |
| PFM-2 | 0.08 | 1.25/0.625 | 160/4 | — |
| PFM-3 | 0.32 | 1.25/0.625 | 160/4 | — |
| PFM-4 | 0.64 | 1.25/0.625 | 160/4 | — |
| PFM-5 | 0.16 | 5/2.5 | 160/4 | — |
| PFM-6 | 0.32 | 10/5 | 160/4 | — |
| PFM-7 | 0.04 | 1.25/0.625 | 160/4 | 10 |

In order to decrease the number of Fe₃O₄ nanoparticles encapsulated in the PF spheres, the Fe₃O₄ magnetic nanoparticles after acid treatment were dispersed in 5 ml of deionized water, then mixed with 5 ml deionized water containing 10 mg sodium oleate at 80° C. The next processes were the same as those mentioned above.

As shown in FIG. 2a-c, when the amount of Fe₃O₄ nanoparticles increases from 0.08 mmol to 0.32 mmol, to 0.64 mmol, the average size of the Fe₃O₄@PF nanospheres decreases from 330 nm to 210 nm to 175 nm. Noticeably, the outer polymer layer is quite uniform with thicknesses of 115 nm, 65 nm, 40 nm, respectively. The average size of multi-core spheres of the three samples ranges from 80-100 nm.

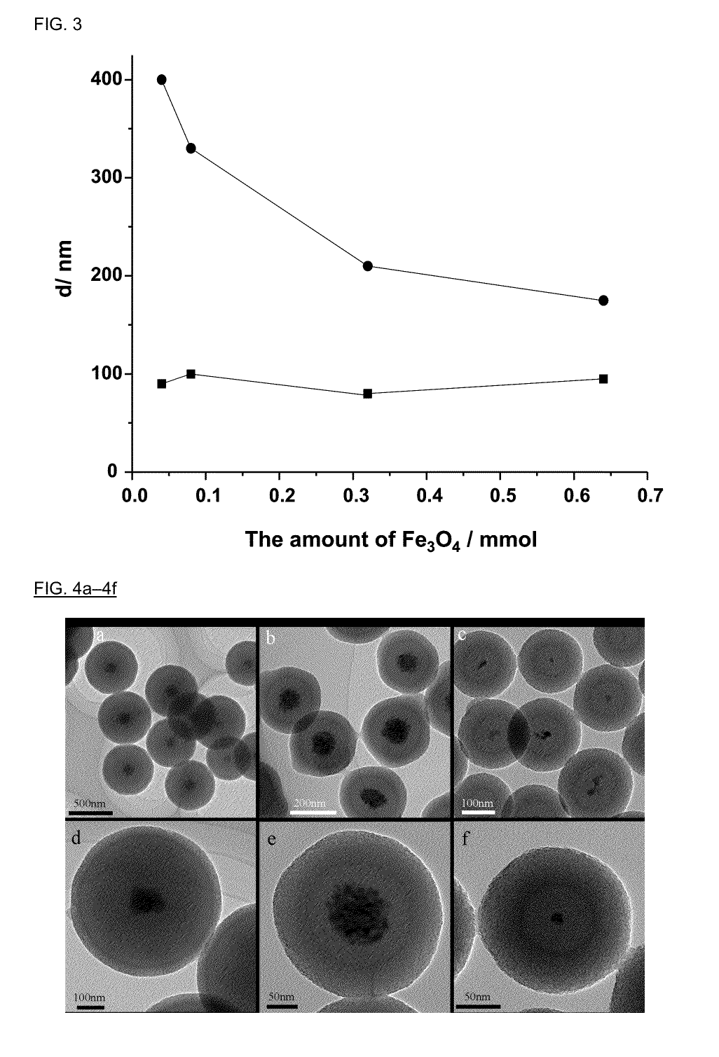

FIG. 3 confirms that the size of multi-core spheres is nearly constant when the amount of Fe₃O₄ nanoparticles changes from 0.04 to 0.64 mmol, but the thickness of the polymer layer decreases obviously. If the amount of Fe₃O₄ nanoparticles further increases to 1.28 mmol, the products become irregular.

### 1.3 Synthesis of Fe₂O₄@PF@SiO₂ Nanospheres

This process was performed according to the procedure described by H. Blas. Briefly, a certain amount of Fe₃O₄@PF nanospheres (120 mg PFM-1, 50 mg PFM-4, and 40 mg PFM-8) was dispersed in 24 ml of ethanol containing 9.84 ml dilute ammonia solution (1.5 M) to form a latex. The surfactant (CTAB, 0.384 g) was stirred with 12 ml of deionized water for 1 h at room temperature. This solution was added to the latex. Then, 52.3 ml of deionized water was added to the mixed solution. The mixture was vigorously stirred for 30 min before adding dropwise 0.575 ml of TEOS over a short period of time. The reaction was carried out at room temperature for 16 h. The TEOS/CTAB/NH₃/ethanol/H₂O molar ratio used in the process was 0.83:0.34:5.3:168:1320. The products were separated by centrifugation and washed several times by deionized water and pure ethanol, and finally dried in an oven at 50° C. for 8 h. The final products were denoted as PFM-x@SiO₂ (see FIG. 4).

### 1.4 Synthesis of Fe₃O₄@Carbon Nanospheres

The Fe₃O₄@PF@SiO₂ composites were heated to 150° C. for 2 h under a nitrogen atmosphere, then heated to the desired temperature (500-800° C.) with a heating ramp of 5° C./min and maintained at this temperature for 2 h to obtain the carbon products. The dissolution of the silica layers was performed in the 2 M NaOH alcohol-water solution (volume ratio of alcohol to water was 1:3) for 24 h. The final products were denoted as PFM-x-y, where y indicates the carbonization temperature.

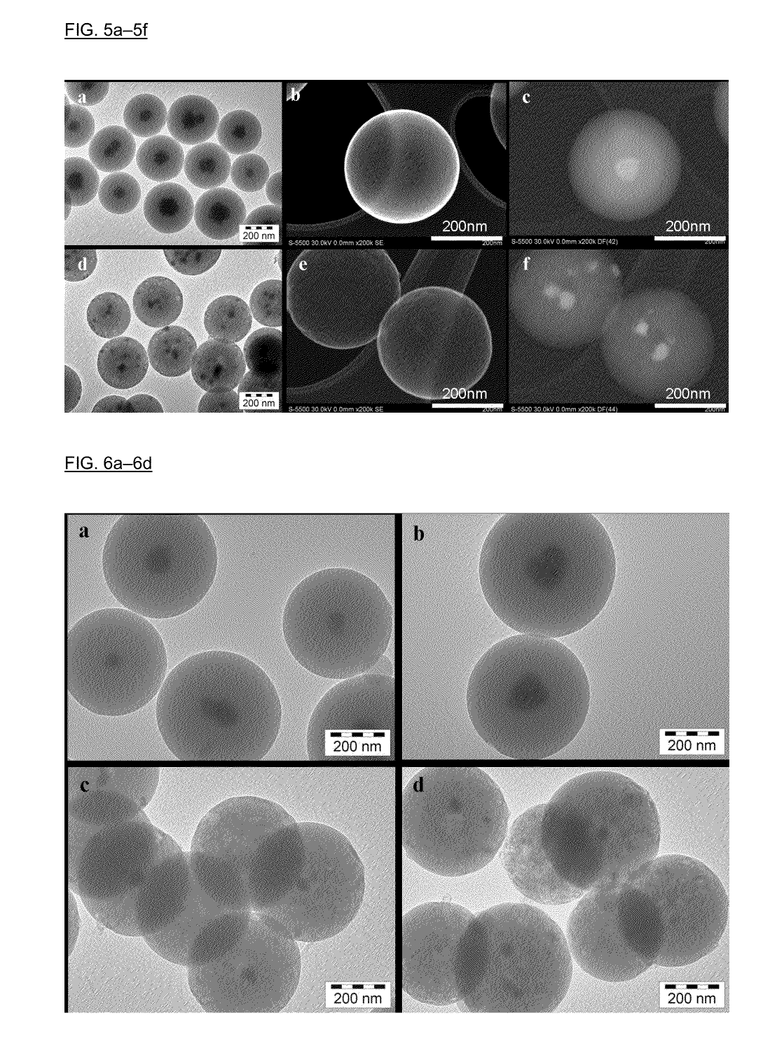

The experiment shows that the Fe₃O₄@PF nanospheres with a diameter larger than 300 nm will not aggregate in the carbonization process. So, the sample PFM-1 was carbonized directly. The morphology of PFM-1-600 and PFM-1-800 were characterized by TEM and STEM analysis. As shown in FIGS. 5a-c, the morphology of sample PFM-1-600 was not changed compared to sample PFM-1, and the Fe₃O₄ multi-core spheres are still located at the center of the carbon spheres. However, the thickness of the outer layers of the Fe₃O₄ multi-core spheres shrinks after carbonization at 600° C., from 155 nm to 125 nm. When the carbonization temperature increases to 800° C., the products with a diameter of ˜350 nm are still monodisperse and uniform; the core of the composite separates into several parts, some of which have moved onto the surface of the composite (as shown in FIGS. 6d-f). Moreover, it can be seen that the products show graphitic layers, which results from the action of the magnetite nanoparticles as graphitization catalysts.

The textural parameters of the products are listed in Table 2. As can be seen, the surface area and pore volume of PFM-1-500 are 470 m²/g and 0.26 cm³/g, respectively. When the carbonization temperature is 600° C., the surface area and pore volume of PFM-1-600 increase to 566 m²/g and 0.3 cm³/g, indicating the generation of much more abundant porosity. With further increase in the carbonization temperature to 700° C., the surface area shows a clearly decreasing trend, but the total pore volume stays almost constant. This is attributed to the conversion of amorphous carbon into graphitic carbon, which destroys the microposity and generates much more mesopores (as shown in the Table, the micropore surface area decreases from 472 to 239 m²/g, and the mesopore volume increases from 0.08 to 0.19 cm³/g). When the carbonization temperature is 800° C., the surface area of PFM-1-800 still decreases, due to further destruction of the microposity.

TABLE 2
Textural parameters of PFM-1-500, PFM-1-600,
PFM-1-700 and PFM-1-800^{a}

| | S_{BET} | S_{mic} | Vₜₒₜₐₗ | V_{mic} | Vₘₑₛₒ |
|---|---|---|---|---|---|
| Samples | (m² · g⁻¹) | (m² · g⁻¹) | (cm³ · g⁻¹) | (cm³ · g⁻¹) | (cm³ · g⁻¹) |
| PFM-1-500 | 470 | 349 | 0.26 | 0.16 | 0.10 |
| PFM-1-600 | 566 | 472 | 0.30 | 0.22 | 0.08 |
| PFM-1-700 | 416 | 239 | 0.30 | 0.11 | 0.19 |
| PFM-1-800 | 374 | 184 | 0.29 | 0.08 | 0.21 |

^{a}S_{BET}: apparent surface area calculated by BET method. S_{mic}: micropore surface area calculated by t-plot method. Vₜₒₜₐₗ: total pore volume at p/p₀ = 0.97. V_{mic}: micropore volume calculated by t-plot method. Vₘₑₛₒ: mesopore volume.

The TEM images in FIG. 6 clearly show that samples PFM-1-500, PFM-1-600, PFM-1-700 and PFM-1-800 are stable against concentrated hydrochloric acid. The magnetic cores are retained, indicating the good stability of these samples.

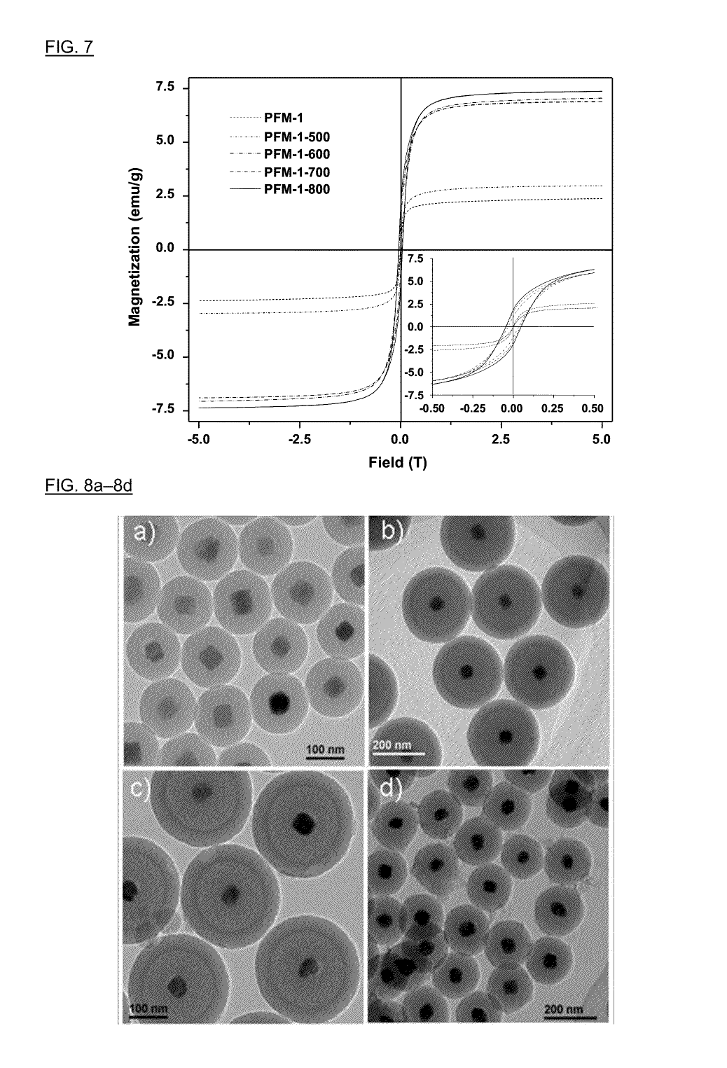

The magnetization curves in FIG. 7 show that these materials are essentially superparamagnetic in nature.

### 2. Examples for Fe₂O₃ Based Nanoparticles

### 2.1 Synthesis of Quasicubic α-Fe₂O₃ Nanoparticles

The quasicubic α-Fe₂O₃ nanoparticles were prepared according to the literature. In a typical experiment, 1.212 g of Fe(NO₃)₃.9H₂O and 1.8 g of PVP were dissolved in 108 ml of N,N-dimethylformamide (DMF). The solution was then turned into a Teflon-lined stainless steel autoclave of 120 ml capacity. The sealed autoclave was put into an oven and heated at 180° C. for 30 h. After reaction, the autoclave was cooled to room temperature naturally. The red precipitates were collected by centrifugation, washed with deionized water and ethanol several times, and redispersed in water.

### 2.2 Synthesis of Fe₂O₃@PF Nanoparticles

In a typical synthesis, the as-prepared Fe₂O₃ (50 mg) nanoparticles were well dispersed in 120 ml water by ultrasonication for 10 min and subsequently a mixture of 3 mmol phenol (P) and 1.5 mmol hexamethylenetetramine (HMT) aqueous solution was added. After ultrasonication for another ten minutes, the solution was transferred into a Teflon-lined autoclave of 120 ml and heated to 160° C., and maintained for 4 h. The system was then allowed to cool to room temperature. The orange precipitates were collected by centrifugation, washed with deionized water and ethanol several times in sequence, and dried in air at 50° C. for 24 h.

### 2.3 Synthesis of Fe₂O₃@PF@MSiO₂ Nanoparticles

The silica shells were grown on the surface of the Fe₂O₃@PF hybrid spheres by sol-gel condensation of tetraethoxysilane (TEOS) in the presence of cetyltrimethyl-ammoniumbromide (CTAB). Typically, the surfactant CTAB (0.16 g) was stirred with 5 ml of deionized water for 1 h at room temperature with a magnetic bar. Then this solution was added to a mixture of 50 mg of Fe₂O₃@PF, 25 ml of deionized water, 10 ml of ethanol and 0.4 ml of ammoniac solution (28-30%). The solution was stirred for 30 min before adding dropwise 0.28 ml TEOS over a short period of time. The reaction was carried out at room temperature during 16 h. Finally, the sample was collected by centrifugation, washed with deionized water and ethanol in sequence, and dried in air at 50° C. for 24 h.

### 2.4 Synthesis of FeₓO_{y}@C Nanoparticles

The synthesis of Fe₂O₃@C nanoparticles involves two steps: carbonization and removal of the silica shell. Firstly, the Fe₂O₃@PF@MSiO₂ was heated at 5 K/min to 150° C., and held at the temperature for 1 h under flowing nitrogen. Then temperature was increased to 500° C., 600° C., 700° C., or 800° C. at a heating rate of 5K/min, respectively, and maintained at that temperature for 2 h. The dissolution of the silica shells using 2N NaOH in a 8:1 mixture of deionized water and ethanol generated the FeₓO_{y}@C nanoparticles. FIG. 8 shows the TEM images of the polymer coated Fe₂O₃@PF, silica coated Fe₂O₃@PF@SiO₂, pyrolyzed Fe₂O₃@PF@SiO₂ and the final target product FexOy@C.

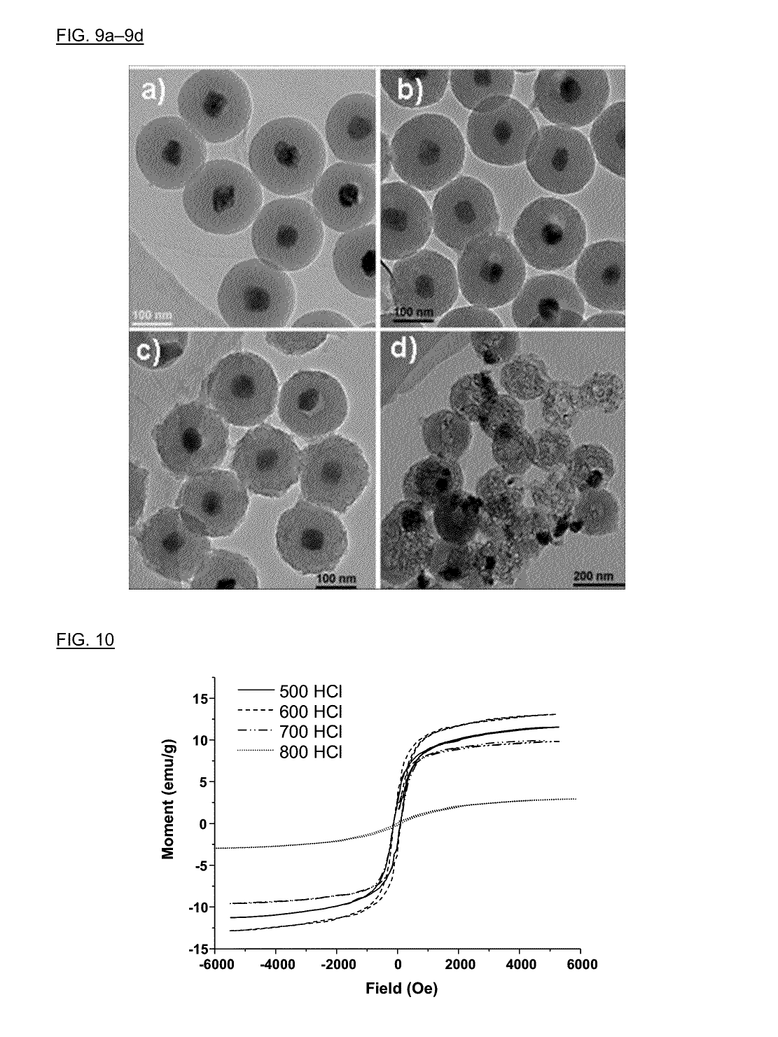

In FIG. 9, the TEM images show the structural details of the FeₓO_{y}@C samples pyrolyzed at different temperatures, 500° C., 600° C., 700° C., and 800° C.

The magnetic properties of FeₓO_{y}@C nanoparticles obtained at different pyrolysis temperatures and after washing with concentrated HCl was measured at room temperature. As shown in FIG. 10, all samples exhibit a typical ferromagnetic behaviour with a hysteresis loop, and the saturation magnetization of the samples ranges from 13.2 emu g⁻¹ to 2.81 emu g⁻¹. It reveals that FeₓO_{y} nanoparticles were well protected by the carbon against leaching by concentrated HCl.

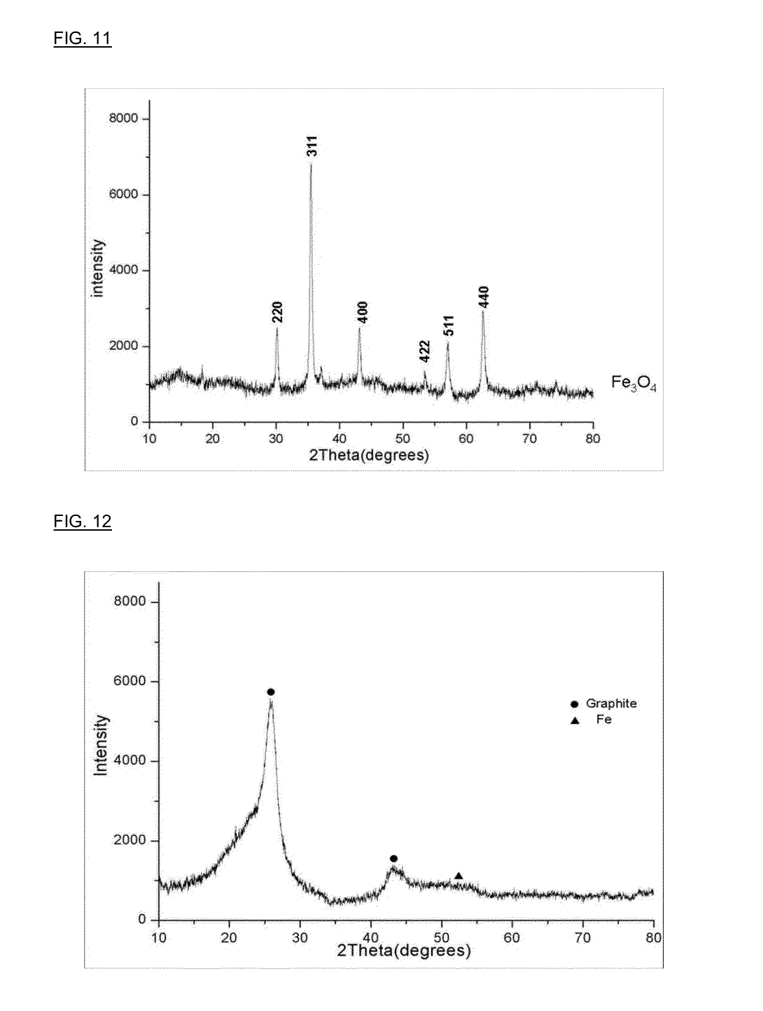

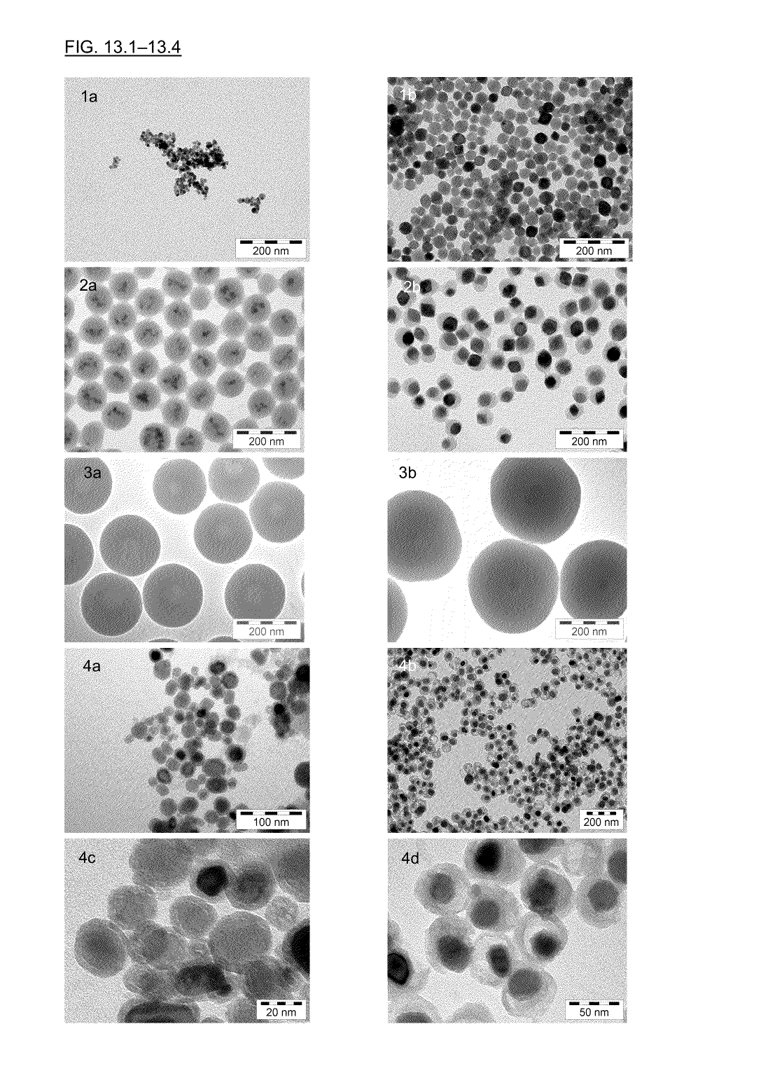

The XRD pattern in FIG. 11 shows that the sample FeₓO_{y}@C pyrolyzed at 600° C. has a magnetite core. The XRD pattern in FIG. 12 shows that sample FeₓO_{y}@C pyrolyzed at 800° C. has a shell with graphitic structure.

### 3. Examples for Fe₃O₄ and Fe₂O₃ Based Nanoparticles

3.1 Synthesis of Water Dispersable Fe₃O₄ Nanoparticles Functionalized with 17-Heptadecenoic Acid: (Fe₃O₄@HDA)

Colloidal Fe₃O₄ nanoparticles with diameters of 10 nm were prepared using a modification of the procedure originally described by R. Massart, V. Cabuil, J. Chem. Phys. 1987, 84, 1247 based on the co-precipitation of iron (II) and iron (III) chlorides in base solution. All steps were performed under argon. In a typical process 5.0 mmol FeCl₃.6H₂O and 2.5 mmol FeCl₂.4H₂O were dissolved in 10 ml H₂O. This solution was injected drop wise into 31.5 ml ammonia solution (1.3% NH₃ in water) at 90° C. under vigorous mechanical stirring. After 30 min the formed black material was collected with a magnet to remove the supernatant. The stabilization of the iron oxide particles in aqueous media was then achieved by adding a mixture of 0.7 mmol 17-heptadecenoic acid dissolved in 5.0 ml ammonia solution (1.3% NH₃ in water). After 1 h stirring at 50° C. a stable dispersion was received. Finally, the black fluid was divided into six equal parts each with a magnetite mass content of about 100 mg.

3.2 Synthesis of Water Dispersable α-Fe₂O₃ Nanoparticles Functionalized with 17-Heptadecenoic Acid (Fe₂O₃@HDA)

2 mmol FeCl₃.6H₂O and 2 mmol L-lysine were dissolved in 100 ml millipore water (18.2 M/cm⁻²). The solution was heated up to 175° C. in an autoclave with a total volume of 110 cm³. The size of the α-Fe₂O₃ crystals was controlled by the heating time. For example, to prepare 35 nm sized particles the autoclave was cooled down after 50 min reaction time. The resultant colloids were centrifuged (14 000 rpm; 12 min) and were washed twice with water. To functionalize the iron oxide surfaces, 100 mg of α-Fe₂O₃ nanoparticles were dispersed in 15 ml ammonia solution (1.3% NH₃ in water) containing 0.445 mmol 17-heptadecenoic acid. The immobilization of the surfactant was performed by ultrasonication for ½ h at 50° C.

### 3.3 Coating of the Iron Oxide Nanoparticles in Cross Linked Polystyrene Spheres:

To coat 100 mg of the functionalized iron oxide nanoparticles in cross-linked polystyrene shells 23.56 mmol styrene, 5.89 mmol divinylbenzene and 2.32 mmol glycidyl methacrylate were added to the dispersion. After 1 h moderate mechanical stirring at 50° C. the reaction mixture was diluted in 142 ml warm ammonia solution (1.3% NH₃ in water) and was then heated up to 70° C. for 20 min. By adding 0.17 mmol NH₄S₂O₈ dissolved in 2 ml H₂O, the polymerization reaction was allowed to proceed for 20 h at constant stirring. Coated Fe₂O₃ nanoparticles (Fe₂O₃@Psty) were separated afterwards from smaller pure polymer spheres by centrifugation while the coated Fe₃O₄ nanoparticles (Fe₃O₄@Psty) could by used in the following steps without further purification.

### 3.4 Encapsulation of the Polymer-Coated Iron Oxide Particles in SiO₂

30 mg coated iron oxide nanoparticles were shaken in a solution containing 0.083 mmol tetra-n-butylammonium bromide, 10 ml ethanol and 8 ml ammonia solution (2% NH₃ in water) for 1 h to place cationic charges on the polymer surfaces. Under vigorous stirring, 20.80 ml ethanol, premixed with concentrated ammonia solution (0.81 ml, 28-30% NH₃ in water) were then added, immediately followed by addition of 3.8 ml tetraethylorthosilicate dissolved in 14.30 ml ethanol. The reaction mixture was stirred for 16 h at room temperature. The resultant colloids were centrifuged (10 000 rpm; 10 min) and washed four times with ethanol. In between washing and following centrifugation the solid was redispersed by ultrasonication. Finally, the dispersion was dried for 1 day at 50° C.

3.5 Pyrolysis Under Reducing Atmosphere and Leaching of the SiO₂-Shells with NaOH

The dried colloids were further heated up to 800° C. with 5 K/min under H₂ atmosphere to reduce the iron oxide core and to convert the cross linked polymer into carbon. The temperature was kept for 1 h followed by a slow cooling process to room temperature. The SiO₂ shells were afterwards removed by adding 300 mg of the material in 25 ml aqueous solution containing 22.5 mmol of sodium hydroxide. After 24 h at 50° C., the solid silica species were completely dissolved. The final product was centrifuged (12 000 rpm; 10 min) and washed four times with water to remove the dissolved ions.

1. Process for preparing carbon protected superparamagnetic or magnetic nanospheres comprising the steps:
(A) coating magnetic and/or superparamagnetic nanoparticles with an organic polymer,
(B) coating the product obtained in step (A) with silica,
(C) subjecting the product of step (B) to pyrolysis conditions, and
(D) removing silica.

2. Process according to claim 1, wherein the magnetic or superparamagnetic particles are selected from Fe, Co, Ni, Mn, Pd, Cr, and any compounds and mixtures thereof.

3. Process according to claim 2, wherein the magnetic or superparamagnetic particles are selected from Fe and FeₓO_{y}.

4. Process according to claim 1, wherein the nanoparticles are coated with the polymer by reacting precursor component(s) of the polymer in the presence of the nanoparticles.

5. Process according to claim 4, wherein the polymer is obtained by polycondensation or radical initiated polymerization of the precursor component(s).

6. Process according to claim 4, wherein the precursor of the polymer is selected from aromatic compounds which can be polymerized with aldehydes or other reactive components and from polymer precursors which are suitable for coating surfaces.

7. Process according to claim 6, wherein the aromatic compound is selected from phenol, resorcinol, phloroglucinol, dihydroxybenzoic acid and mixtures thereof.

8. Process according to claim 6, wherein the aldehyde compound is selected from formaldehyde, acetaldehyde, propaldehyde, glutaraldehyde and mixtures thereof.

9. Process according to claim 6, wherein polymer precursors which are suitable for coating surfaces are selected from hexamethylene tetramine, styrene, divinylbenzene(meth)acrylates, glycidyl(meth)acrylate(s), and mixtures of styrene, divinylbenzene, (meth)acrylate and/or glycidyl(meth)acrylate.

10. Process according to claim 1, wherein in step (B) coating with silica is carried out by subjecting one or more precursor compounds of silica to hydrolysis conditions in the presence of the product of step (A).

11. Process according to claim 10, wherein the precursor compound of silica is selected from (R¹O)₄—Si, wherein R¹ is selected from an alkyl group having 1 to 6 carbon atoms.

12. Process according to claim 10, wherein the hydrolysis is carried out under basic conditions.

13. Process according to claim 1, wherein the pyrolysis is carried out at a temperature from 450° C. to 900° C.

14. Process according to claim 1, wherein silica is removed by treating the product of step (C) with a basic solution having a pH value above 10.

15. Process according to claim 14, wherein the solution is an aqueous, ethanolic or aqueous/ethanolic solution of an alkali hydroxide.

16. Nanospheres obtainable according to the process of claim 1.

17. Method of using the nanospheres according to claim 16 as catalyst particles or catalyst supports, in magnetic fluids, for biotechnology/biomedicine processes, as contrast agents for imaging methods and drug targeting.

18. Method of using the nanospheres according to claim 17, wherein the biotechnology/biomedicine processes comprise hyperthermia, separation of biomolecules, enrichment of biomolecules.
